(19) United States
(12) Patent Application Publication
(10) Pub. No.: US 2022/0022706 A1
(43) Pub. Date: Jan. 27, 2022

(54) HAND DRYER

(71) Applicant: MILA INTERNATIONAL INC., Huntington Beach, CA (US)

(72) Inventor: Shujen YU, Huntington Beach, CA (US)

(73) Assignee: MILA INTERNATIONAL INC., Huntington Beach, CA (US)

(21) Appl. No.: 17/292,389

(22) PCT Filed: Nov. 7, 2018
(86) PCT No.: PCT/US18/59632

§ 371 Date: May 7, 2021

(51) Int. Cl. A47K 10/48; F26B 21/00; F26B 21/10; A61L 2/26; A61L 2/10

(52) U.S. Cl. CPC A47K 10/48; F26B 21/004; A61L 2/10; A61L 2/26; F26B 21/10

(57) ABSTRACT

In an implementation, a hand dryer is disclosed. The hand dryer includes an air compressor and a storage tank connected to the air compressor. The storage tank is configured to store pressurized air received from the air compressor. The hand dryer also includes an air manifold connected to the storage tank, and the air manifold includes at least one aperture. The hand dryer also includes a valve for selectively releasing the pressurized air from the storage tank into the air manifold such that the pressurized air is discharged from the at least one aperture.

### INTRODUCTION

The information provided in this section is for the purpose of generally presenting the context of the disclosure. Work of the presently named inventors, to the extent it is described in this section, as well as aspects of the description that may not otherwise qualify as prior art at the time of filing, are neither expressly nor impliedly admitted as prior art against the present disclosure.

The present disclosure relates to a hand dryer, and more particularly to a hand dryer that is configured to release compressed air to dry a user's hand.

Public restrooms typically include hand dryers as an alternative or in addition to paper towels. These hand dryers typically include a motor that generates air flow that is directed to a user's hand to remove water from the hand.

### SUMMARY

In an implementation, a hand dryer is disclosed. The hand dryer includes an air compressor and a storage tank connected to the air compressor. The storage tank is configured to store pressurized air received from the air compressor. The hand dryer also includes an air manifold connected to the storage tank, and the air manifold includes at least one aperture. The hand dryer also includes a valve for selectively releasing the pressurized air from the storage tank into the air manifold such that the pressurized air is discharged from the at least one aperture.

In other features, the hand dryer includes a controller that is connected to the valve and is configured to cause the valve to selectively actuate the valve based upon a proximity signal.

In other features, the hand dryer includes a proximity sensor disposed proximate to the air manifold and is configured to transmit the proximity signal to the controller upon detection of an object proximate to the proximity sensor.

In other features, the controller is further configured to receive a measured air pressure signal and cause the air compressor to discharge air into the storage tank based upon the measured air pressure signal.

In other features, the hand dryer includes an air pressure sensor disposed within the storage tank and configured to measure an air pressure within the storage tank and provide the measured air pressure signal to the controller.

In other features, the hand dryer includes an electromagnetic radiation source that is configured to emit electromagnetic radiation.

In other features, the electromagnetic radiation source is disposed proximate to the at least one aperture of the hand dryer.

In other features, the electromagnetic radiation source is configured to emit ultraviolet (UV) electromagnetic radiation.

In other features, the controller is further configured to cause the electromagnetic radiation source to emit ultraviolet (UV) electromagnetic radiation based upon the proximity signal.

In other features, the hand dryer includes the controller is further configured to cause the electromagnetic radiation source to emit ultraviolet (UV) electromagnetic radiation based upon the proximity signal.

In other features, the at least one aperture comprises a linear slot.

In an implementation, a hand dryer system is described. The hand dryer system includes an air compressor and a storage tank connected to the air compressor. The storage tank is configured to store pressurized air received from the air compressor. The hand dryer system also includes a plurality of hand dryers connected to the air compressor. Each hand dryer of the plurality of hand dryers configured to release pressurized air through at least one linear slot, and the pressurized air is received from the storage tank. The hand dryer system also includes a plurality of valves, where the respective valves of the plurality of valves disposed between each hand dryer of the plurality of hand dryers and the storage tank.

In other features, each hand dryer of the plurality of hand dryers further includes a controller configured to control release of pressurized air from the storage tank based upon a proximity signal.

In other features, each hand dryer of the plurality of hand dryers further includes a proximity sensor disposed proximate to an air manifold of the respective hand dryer. The proximity sensor is configured to transmit the proximity signal to a controller of the respective hand dryer upon detection of an object proximate to the proximity sensor.

In other features, the air compressor includes a controller. The controller of the air compressor is further configured to receive a measured air pressure signal and cause the air compressor to discharge air into the storage tank based upon the measured air pressure signal.

In other features, the hand dryer system includes an air pressure sensor disposed within the storage tank, and the air pressure sensor is configured to measure an air pressure within the storage tank and provide the measured air pressure signal to the controller of the air compressor.

In other features, each hand dryer of the plurality of hand dryers further comprises an electromagnetic radiation source that is configured to emit electromagnetic radiation.

In other features, the electromagnetic radiation source is disposed proximate to an aperture of the respective hand dryer.

In other features, the electromagnetic radiation source is configured to emit ultraviolet (UV) electromagnetic radiation.

In other features, the controller of the respective hand dryer is further configured to cause the electromagnetic radiation source to emit ultraviolet (UV) electromagnetic radiation based upon the proximity signal.

In other features, the controller of the air compressor is connected to the plurality of valves and is configured to selectively actuate the plurality of valves based upon the proximity signal.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

### BRIEF DESCRIPTION OF THE DRAWINGS

The detailed description is described with reference to the accompanying figures. The use of the same reference numbers in different instances in the description and the figures may indicate similar or identical items.

FIG. 1 is a block diagram of a hand dryer in accordance with an example implementation of the present disclosure.

FIG. 2 is a perspective view of a hand dryer in accordance with an example implementation of the present disclosure.

FIG. 3 is a diagrammatic illustration of a hand dryer in accordance with an example implementation of the present disclosure.

FIG. 4 is a perspective view of a hand dryer in accordance with an example implementation of the present disclosure.

FIG. 5 is a diagrammatic illustration of a hand dryer in accordance with an example implementation of the present disclosure.

FIG. 6 is a diagrammatic illustration of a hand dryer in accordance with an example implementation of the present disclosure.

FIG. 7 is a block diagram of a hand dryer system in accordance with an example implementation of the present disclosure.

FIG. 8 is flow diagram of an example method for controlling air discharged from an air compressor to a storage tank of a hand dryer in accordance with an example implementation of the present disclosure.

FIG. 9 is flow diagram of an example method for controlling air discharged from an air manifold of a hand dryer in accordance with an example implementation of the present disclosure.

FIG. 10 is flow diagram of an example method for controlling a hand dryer system in accordance with an example implementation of the present disclosure.

### DETAILED DESCRIPTION

Electric hand dryers have been used within public restrooms or washrooms for some time—but all models on the market currently have various degrees of dysfunctionality. Some do not work well and others do not work at all in the real world sense. For example, after user's put their hands under or into one of the various types of hand dryers produced on the market, the user finds after a few seconds that his/her hands are still wet. Instead of waiting the minimum time of 10 seconds or more that is required to dry ones hand the user will give up, or reach for a paper towel if available. In this sense, all current hand dryers on the market fail to do the job they are designed to do and do not provide any positive environmental impact by saving paper waste as most claim.

Theoretically in order for a hand dryer to really “work,” it is contemplated that the hand dryer should dry a user's hands within 1 to 3 seconds. It has been found that a hand dryer should meet these specifications to be functional in real world usage as any longer than 4 or 5 seconds of usage will cause a user to lose patience and give up. A hand dryer with functional drying time of 1 to 3 seconds will entice usage by the general public, resulting in less usage of paper and true positive environmental impact.

It has been found that current hand dryers on the market do not work because of deficiencies in their technology. Most hand dryers are manufactured with the same basic technology: a blower driven by motor force air through nozzles or pin holes. These hand dryers generally take around 10 to even 20 seconds to dry hands. Hence, these hand dryers do not work in the real world as many people are unwilling to stand in front of a hand dryer for 10 to 20 seconds or more. Without faster dry time, electric hand dryers provide no real value to the public spaces that such units are installed, and often fail to fully alleviate the waste of paper products, which is the exact problem that electric hand dryers claim to solve. In this sense, the current hand dryers on the market offer no real value to users, nor do they have the environmental benefit that many of these hand dryer manufacturers claim to provide. As mentioned above, the hand dryers force air through pin holes—not linear slots. The present disclosure is directed to a hand dryer that uses a compressed air technology to dry hands that is different from the traditional hand dryer design. By using high pressurized air and laminar airflow technology, effective hand drying can be achieved in a rapid time (e.g., approximately 3 seconds or less). The present disclosure may also include linear slots to generate a true blade of air that removes water from a hand.

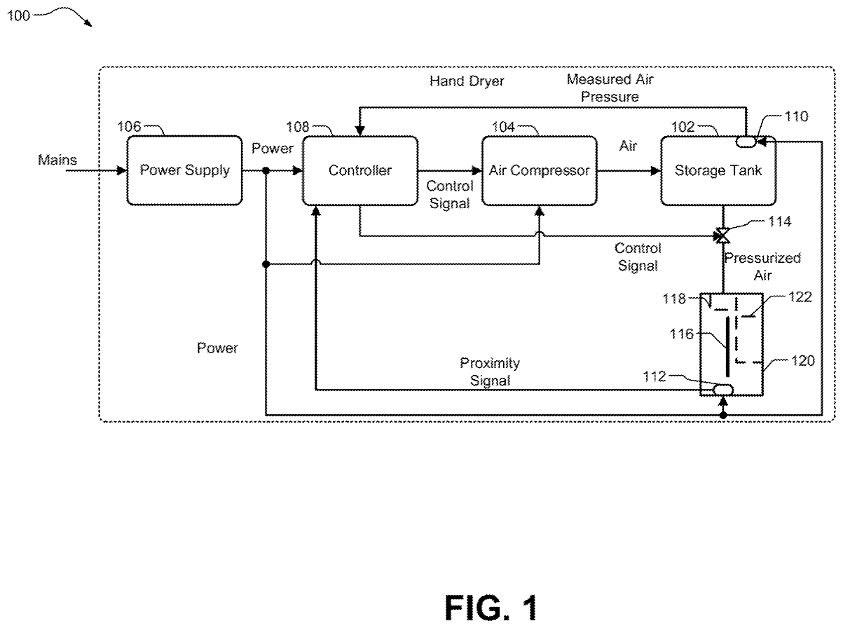

FIG. 1 illustrates a block diagram of an example hand dryer 100 in accordance with an example implementation of the present disclosure. As shown, the hand dryer 100 includes a storage tank 102, an air compressor 104, a power supply 106, a controller 108, an air pressure sensor 110, a proximity sensor 112, and one or more valves 114. The hand dryer 100 discharges airflow to dry a user's hands. In some implementations, the hand dryer 100 is configured to discharge airflow at a high speed (>80 meters/second) through a plurality of apertures 116, which are described in greater detail below. More specifically, the discharge airflow may exceed greater than 110 meters/second in some implementations. In some implementations, the hand dryer 100 may include a regulator 114 that regulates the air flow exiting through the apertures 116.

The power supply 106 may be configured in a variety of ways. For example, the power supply 106 may be connected to mains electricity. The power supply 106 powers one or more of the components of the hand dryer 100. For example, the power supply 106 can provide power the air compressor 104, the controller 108, the air pressure sensor 110, and the proximity sensor 112.

The storage tank 102 is connected to the air compressor 104 and stores pressurized air received from the air compressor 104. The air compressor 104 may comprise any suitable air compressor that displaces air into the storage tank 102 until an air pressure threshold is reached. The air pressure within the storage tank can be detected by the air pressure sensor 110 that is disposed within the storage tank 102.

The controller 108 is connected to the air compressor 104, the air pressure sensor 110, the proximity sensor 112, and the valves 114. During operation, the controller 108 compares the measured air pressure with the air pressure threshold and determines whether the measured air pressure exceeds the air pressure threshold. The controller 108 ceases operation of the air compressor 104 when the air pressure threshold is exceeded. Upon receiving a signal from the proximity sensor 112 indicating that an object is proximate to the hand dryer 100, the controller 108 causes the valves 114 to actuate to release pressurized air from the storage tank 102. The pressurized air is provided to an air manifold 120, and the compressed, or pressurized, air is dispersed through the apertures 116. As shown, the apertures 116 comprise narrow, slit-like nozzles (e.g., linear slots). For example, compressed air is forced through these narrow, slit-like nozzles to generate thin sheets (“blades”) of high-velocity air that remove water from the user's hands. In an example implementation, the narrow, like-like nozzles may be less than one (1) millimeter wide.

Once the measured air pressure goes below the air pressure threshold, the air compressor 104 provides compressed air to the storage tank 102 until the air pressure threshold is exceeded. In implementations where the hand dryer 100 includes the regulator 118, the regulator 118 can regulate the air pressure prior to the air being directed outwardly via the apertures 116. For example, the regulator 118 may further increase the pressure of the pressurized air or decrease the pressure of the pressurized air.

In some implementations, the hand dryer 100 may further include an electromagnetic radiation source 122. The electromagnetic radiation source 122 may be disposed within the air manifold 120. The electromagnetic radiation source 122 can be connected to the controller 108 and is configured to generate electromagnetic radiation to mitigate germs and bacteria during the hand drying process and/or sanitary benefits. For example, the electromagnetic radiation source 122 can generate ultraviolet (UV) light. The UV light can be generated when the controller 108 receives the proximity signal indicative of the object proximate to the hand dryer 100. The controller 108 may also cause the electromagnetic radiation source 122 to generate UV light at predetermined time periods (e.g., every 1 hour, every 2 hours, etc.) for sanitary purposes, such as reducing bacteria and/or germ buildup within the hand dryer 100.

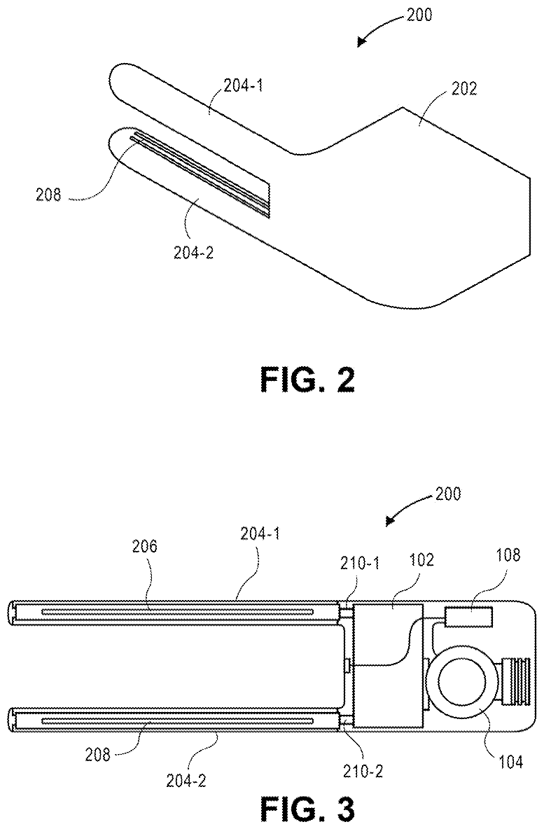

FIGS. 2 and 3 illustrate an example hand dryer 200 in accordance with an example implementation of the present disclosure. The hand dryer 200 includes an external housing 202, or casing, that retains the components of the hand dryer 100. In this implementation, the hand dryer 200 includes a first air manifold 204-1 and a second air manifold 204-2. The first air manifold 204-1 includes a first set of air apertures 206 defined therein and the second air manifold 204-2 includes a second set of air apertures 208 defined therein. In one or more implementations, the apertures 206, 208 can be formed into the housing 202 via suitable machining methods. It is understood that the apertures may be configured in a variety of ways. For example, the apertures may be machined to promote additional airflow while the airflow is being discharged (i.e., increasing the effective discharge rate of air from the apertures). The storage tank 102 is connected to the air manifolds 204-1, 204-2 via passages 210-1, 210-2. The passages 210-1, 210-2 can comprise any suitable pipes, or the like.

During operation, the pressurized air released from the storage tank 102 has a higher flow rate that creates a boost of airflow that expedites drying time by blowing the water off of the user's hand. The air manifolds 204-1, 204-2 can be parallel to one another such that a hand inserted between the air manifolds 204-1, 204-2 will receive air blown out of the first set of air apertures 206 and out of the second set of air apertures 208. In an example implementation, the first set of apertures 206 and the second set of apertures 208 may be aligned with one another. For example, each aperture of the first set of apertures 206 may be aligned with a corresponding aperture of the second set of apertures 208.

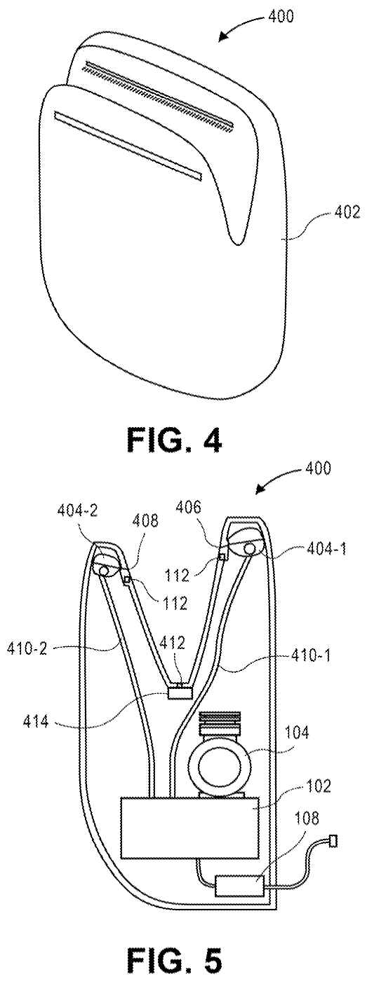

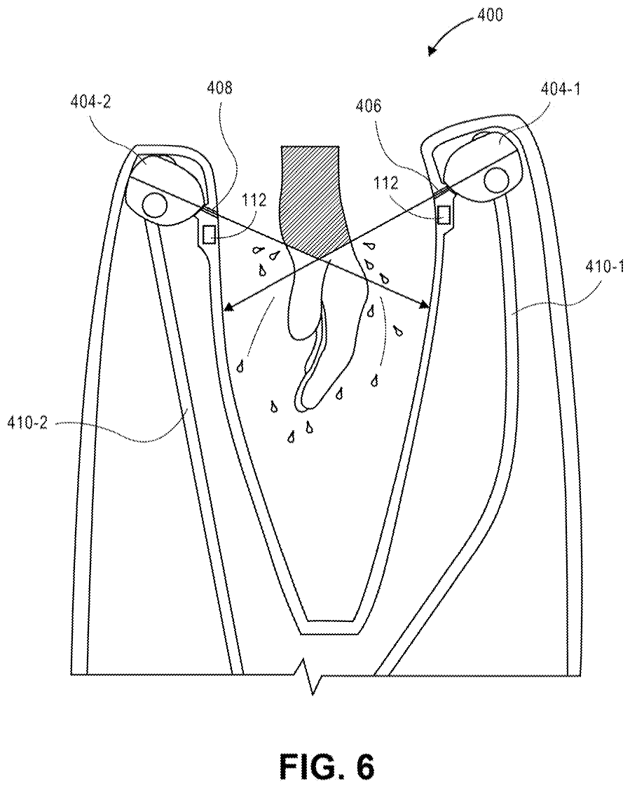

FIGS. 4 through 6 illustrate another example hand dryer 400 according to an example implementation of the present disclosure and operates similar to the hand dryer 200 described above. As shown, the hand dryer 400 includes an external housing 402 that retains the components of the hand dryer 400. In this implementation, the hand dryer 400 includes a first air manifold 404-1 and a second air manifold 404-2. The first air manifold 404-1 includes a first set of air apertures 406 defined therein and the second air manifold 404-2 includes a second set of air apertures 408 defined therein. The storage tank 102 is connected to the air manifolds 404-1, 404-2 via passages 410-1, 410-2.

In one or more examples, the hand dryers 200, 400 may include a drain 412 and an excess water receptacle 414. The drain 412 may be machined within the housing 202, 402, and the excess water receptacle 414 may be a detachable receptacle that retains water blown off of the hand.

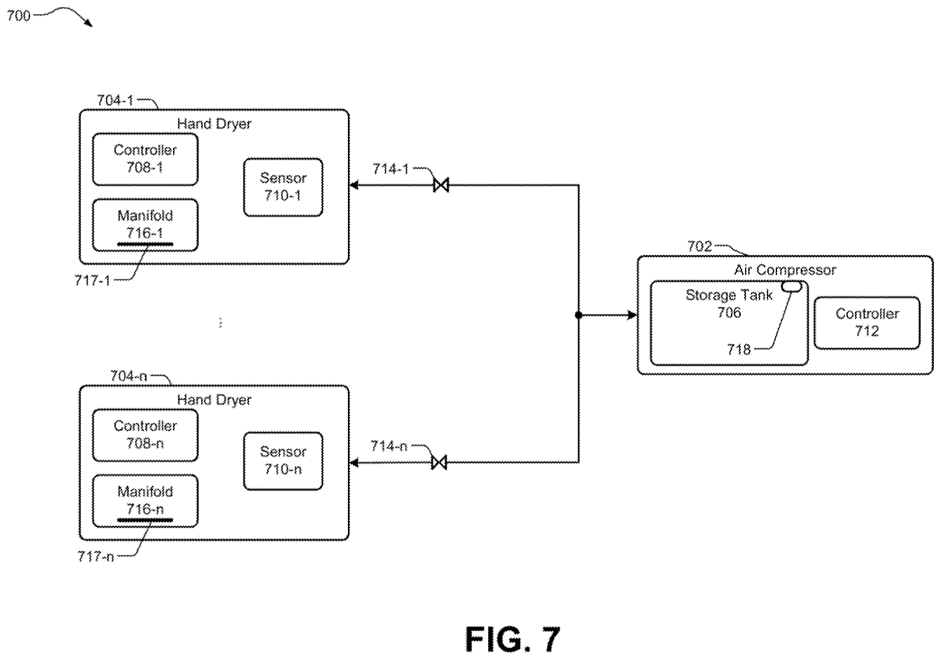

FIG. 7 illustrates an example system 700 according to an example implementation of the present disclosure. As shown, the system 700 includes an air compressor 702 that is connected to multiple hand dryers 704-1 to 704-n. The air compressor 702 includes a storage tank 706. Each hand dryer 704-1 to 704-n includes a respective controller 708-1 to 708-n that receives proximity signals from proximity sensors 710-1 to 710-n. The proximity signal is indicative of whether an object, such as a hand, is proximate to the hand dryer 704-1 to 704-n.

The controllers 708-1 to 708-n send a control signal to the v indicating whether pressurized air is needed based upon a detected proximity signal. The air compressor 702 may also include a controller 712 that controls operation of the air compressor 702. The controller 712 may also control actuation of one or more valves 714-1 to 714-n. The valves 714-1 to 714-n may be used to control the flow of pressurized air within the system 700. For example, when the controller 712 receives a proximity signal from the controller 708-1, the controller 712 causes the valve 714-1 to actuate to allow the flow of pressurized air from the storage tank 706 while maintaining the other valves closed. Pressurized air is provided to a respective air manifold 716-1 to 716-n based upon one or more proximity signals and released through respective apertures to remove water from the object. The valves 714-1 to 714-n may also regulate the pressurized air released through the air manifolds 716-1 to 716-n. The pressurized air is released through respective 717-1 to 717-n apertures (e.g., nozzles, linear slots, etc.).

As shown in FIG. 7, the system 700 also includes an air pressure sensor 718 that measures the air pressure within the storage tank 706. The controller 712 can regulate the amount of pressurized air within the storage tank 706. For example, when pressurized air is released to one of the air manifolds 716-1 to 716-n, the controller 712 can control the air compressor 702 to provide pressurized air to the storage tank 706 until a predetermined threshold is reached to replenish the pressurized air stored in the storage tank 706. The system 700 can include a suitable number of hand dryers 704-1 to 704-n that are connected to the air compressor 702. The number of hand dryers 704-1 to 704-n is based upon the size of the air compressor 702. In some implementations, the system 700 may include up to five (5) hand dryers 704. However, it is understood that the system 700 can include more or less hand dryers 704.

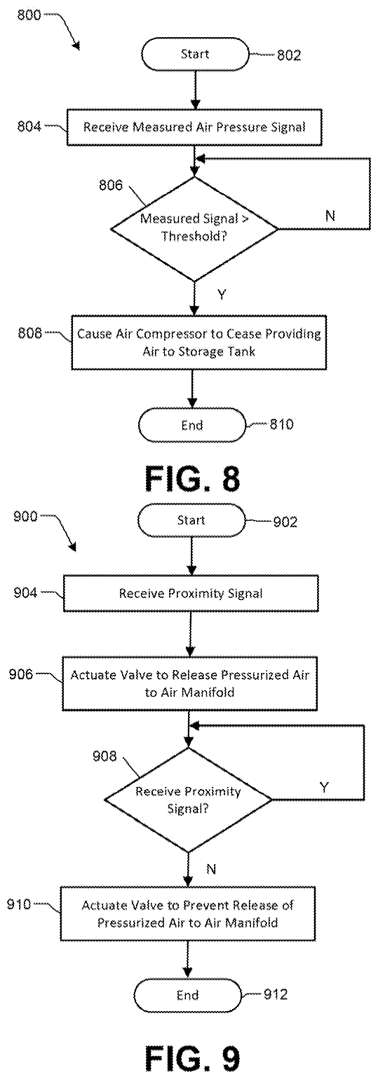

FIG. 8 illustrates an example method 800 for controlling a hand dryer, such as hand dryer 100, according to an example implementation of the present disclosure. The method 800 begins at 802. At 804, the controller 108 receives a measure air pressure signal from the air pressure sensor 110 indicative of the air pressure within the storage tank 102. At 806, the controller 108 determines whether the measured air pressure exceeds a predetermined air pressure threshold.

If the measured air pressure does not exceed the predetermined air pressure threshold, the method returns to 806. At 808, the controller 108 sends a control signal to the air compressor 104 to cause the air compressor 104 to cease providing additional air to the storage tank 102. The method 800 ends at 810.

FIG. 9 illustrates an example method 900 for controlling a system, such as system 700, according to an example implementation of the present disclosure. The method 1000 begins at 1002. At 1004, the controller 108 receives a proximity signal from the proximity sensor 112 indicating an object, such as a hand, is proximate to the proximity sensor 112. At 906, the controller 108 sends a control signal to the valve 114 to actuate the valve 114 such that pressurized air is released to the air manifold 120. At 908, the controller 108 determines whether the proximity signal is still being received. If the proximity signal has not been received, the controller 108 sends another control signal to the valve 114 to prevent the release of pressurized air from the storage tank 102 at 910. If the proximity signal is received, the method 900 returns to 908. The method 900 ends at 912.

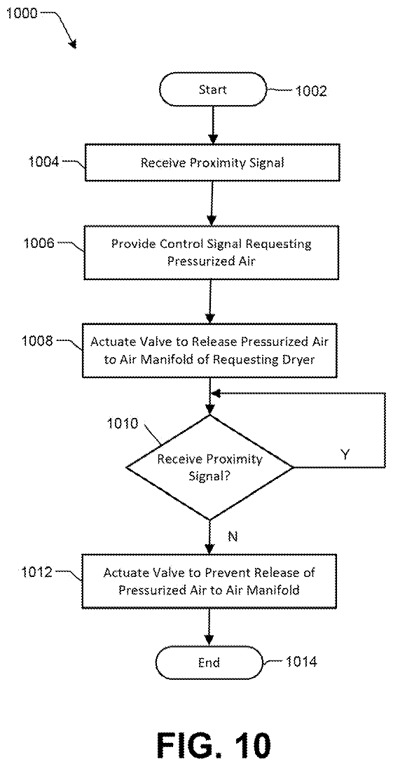

FIG. 10 illustrates an example method 900 for controlling a hand dryer, such as hand dryer 100, according to an example implementation of the present disclosure. Method 900 is described with respect to hand dryer 704-1 for simplicity purposes. However, it is understood that any of the hand dryers within the system 700 may be used within the described context.

The method 1000 begins at 1002. At 1004, the controller 708-1 receives a proximity signal from the proximity sensor 710-1 indicating an object, such as a hand, is proximate to the proximity sensor 710-1. At 1006, the controller 708-1 sends a control signal to the controller 712 requesting pressurized air based upon the proximity signal. At 1008, the controller 712 actuates the valve 714-1 to cause pressurized air to be released from the storage tank 706 and provided to the air manifold 716-1.

At 1010, the controller 708-1 determines whether the proximity signal is still being received. If the proximity signal has not been received, the controller 708-1 sends another control signal to the controller 712 to prevent the release of pressurized air from the storage tank 102 at 1012. For example, the controller 712, upon receiving the control signal indicating no proximity signal is still being received, the controller 712 actuates the valve 714-1 to prevent further release of pressurized air from the storage tank 706. If the proximity signal is received, the method 1000 returns to 1010. The method 1000 ends at 1014.

The foregoing description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. It should be understood that one or more steps within a method may be executed in different order (or concurrently) without altering the principles of the present disclosure. Further, although each of the implementations is described above as having certain features, any one or more of those features described with respect to any implementation of the disclosure can be implemented in and/or combined with features of any of the other implementations, even if that combination is not explicitly described. In other words, the described implementations are not mutually exclusive, and permutations of one or more implementations with one another remain within the scope of this disclosure.

Spatial and functional relationships between elements (for example, between modules, circuit elements, semiconductor layers, etc.) are described using various terms, including “connected,” “engaged,” “connected,” “adjacent,” “next to,” “on top of,” “above,” “below,” and “disposed.” Unless explicitly described as being “direct,” when a relationship between first and second elements is described in the above disclosure, that relationship can be a direct relationship where no other intervening elements are present between the first and second elements, but can also be an indirect relationship where one or more intervening elements are present (either spatially or functionally) between the first and second elements. As used herein, the phrase at least one of A, B, and C should be construed to mean a logical (A OR B OR C), using a non-exclusive logical OR, and should not be construed to mean “at least one of A, at least one of B, and at least one of C.”

In the figures, the direction of an arrow, as indicated by the arrowhead, generally demonstrates the flow of information (such as data or instructions) that is of interest to the illustration. For example, when element A and element B exchange a variety of information but information transmitted from element A to element B is relevant to the illustration, the arrow may point from element A to element B. This unidirectional arrow does not imply that no other information is transmitted from element B to element A. Further, for information sent from element A to element B, element B may send requests for, or receipt acknowledgements of, the information to element A.

1. A hand dryer, comprising:
an air compressor;
a storage tank connected to the air compressor, the storage tank configured to store pressurized air received from the air compressor;
an air manifold connected to the storage tank, the air manifold having at least one aperture;
a valve for selectively releasing the pressurized air from the storage tank into the air manifold such that the pressurized air is discharged from the at least one aperture;
a proximity sensor disposed proximate the aperture on the air manifold, the proximity sensor configured to detect an object near the aperture; and
a controller configured to actuate the valve to release pressurized air based on a signal from the proximity sensor.

2-20. (canceled)

21. The hand dryer of claim 1, further comprising a housing supporting the air manifold, wherein the housing includes a pair of legs separated by a channel, the channel is configured to receive an object, and the air manifold is supported in one of the pair of legs.

22. The hand dryer of claim 1, further comprising a second air manifold connected to the storage tank, the second air manifold having an aperture, wherein the air manifold is a first air manifold, and the first air manifold and the second air manifold are disposed parallel and aligned vertically on the one of the pair of legs.

23. The hand dryer of claim 1, further comprising a pressure regulator configured to alter a pressure of the pressurized air from the storage tank before the pressurized air is expelled through the aperture in the air manifold, wherein the pressure regulator selectively increases or decreases the pressure to within a range.

24. The hand dryer of claim 1, further comprising an electromagnetic radiation source disposed within the air manifold and configured to generate electromagnetic radiation.

25. A hand dryer, comprising:
a housing;
an air compressor;
a storage tank connected to the air compressor, the storage tank configured to store pressurized air received from the air compressor;
an air manifold connected to the storage tank, the air manifold being disposed in the housing, the air manifold having an aperture, the aperture being a single, elongated slot in the air manifold that extends a length of the housing, the elongated slot having a width of about 1 millimeter (mm); and
a valve for selectively automatically releasing the pressurized air from the storage tank into the air manifold such that the pressurized air is discharged from the at least one aperture.

26. The hand dryer of claim 25, further comprising a housing supporting the air manifold, wherein the housing includes a pair of legs separated by a channel, the channel is configured to receive an object, and the air manifold is supported in one of the pair of legs.

27. The hand dryer of claim 25, further comprising a second air manifold connected to the storage tank, the second air manifold having an aperture, wherein the air manifold is a first air manifold, and the first air manifold and the second air manifold are disposed parallel and aligned vertically on the one of the pair of legs.

28. The hand dryer of claim 25, further comprising a pressure regulator configured to alter a pressure of the pressurized air from the storage tank before the pressurized air is expelled through the aperture in the air manifold, wherein the pressure regulator selectively increases or decreases the pressure to within a range.

29. The hand dryer of claim 25, further comprising an electromagnetic radiation source disposed within the air manifold and configured to generate electromagnetic radiation.

30. A hand dryer, comprising:
an air compressor;
a storage tank connected to the air compressor, the storage tank configured to store pressurized air received from the air compressor, the storage tank being configured to store pressurized air within a pressure range;
an air manifold connected to the storage tank, the air manifold having at least one aperture; and
a valve for selectively releasing the pressurized air from the storage tank into the air manifold such that the pressurized air is discharged from the at least one aperture, the valve being configured to release the pressurized air at a velocity ranging from at least about 80 meters/second (m/s) to about 110 m/s.

31. The hand dryer of claim 30, further comprising a housing supporting the air manifold, wherein the housing includes a pair of legs separated by a channel, the channel is configured to receive an object, and the air manifold is supported in one of the pair of legs.

32. The hand dryer of claim 30, further comprising:
a second air manifold connected to the storage tank, the second air manifold having an aperture, wherein the air manifold is a first air manifold, and the first air manifold and the second air manifold are disposed parallel and aligned vertically on the one of the pair of legs.

33. The hand dryer of claim 30, further comprising a second air manifold connected to the storage tank, the second air manifold having an aperture, wherein the air manifold is a first air manifold, and the first air manifold and the second air manifold are disposed parallel and aligned vertically on the one of the pair of legs.

34. The hand dryer of claim 30, further comprising:
a pressure regulator configured to alter a pressure of the pressurized air from the storage tank before the pressurized air is expelled through the aperture in the air manifold,
wherein the pressure regulator selectively increases or decreases the pressure to within a range.

35. The hand dryer of claim 30, further comprising:
an electromagnetic radiation source disposed within the air manifold and configured to generate electromagnetic radiation.

36. A hand dryer, comprising:
an air compressor;
a storage tank connected to the air compressor, the storage tank configured to store pressurized air received from the air compressor;
an air manifold connected to the storage tank, the air manifold having an aperture, the air manifold having an egg-shaped cross-section and being configured to expel air from the aperture in a rounded point of the egg-shaped cross-section; and
a valve for selectively releasing the pressurized air from the storage tank into the air manifold such that the pressurized air is discharged from the at least one aperture.

37. The hand dryer of claim 36, further comprising:
a housing supporting the air manifold, wherein
the housing includes a pair of legs separated by a channel,
the channel is configured to receive an object, and
the air manifold is supported in one of the pair of legs.

38. The hand dryer of claims 36, further comprising:
a second air manifold connected to the storage tank, the second air manifold having an aperture, wherein
the air manifold is a first air manifold, and
the first air manifold and the second air manifold are disposed parallel and aligned vertically on the one of the pair of legs.

39. The hand dryer of claims 36, further comprising:
a pressure regulator configured to alter a pressure of the pressurized air from the storage tank before the pressurized air is expelled through the aperture in the air manifold,
wherein the pressure regulator selectively increases or decreases the pressure to within a range.

40. The hand dryer of claims 36, further comprising:
an electromagnetic radiation source disposed within the air manifold and configured to generate electromagnetic radiation.
